(19) United States
(12) Patent Application Publication
(10) Pub. No.: US 2003/0180329 A1
(43) Pub. Date: Sep. 25, 2003

(54) Viral vaccine production method

(76) Inventors: Thomas P. Monath, Harvard, MA (US); Farshad Guirakhoo, Melrose, MA (US); Juan Arroyo, S. Weymouth, MA (US)

Correspondence Address: CLARK & ELBING LLP, 101 FEDERAL STREET, BOSTON, MA, 02110, US

(21) Appl. No.: 10/342,681

(22) Filed: Jan. 15, 2003

Related U.S. Application Data
(60) Provisional application No. 60/348,565, filed on Jan. 15, 2002.

(51) Int. Cl. C12Q 1/68; C12P 21/04; A61K 39/12; A61K 39/193

(52) U.S. Cl. 424/218.1; 435/6; 435/69.7

(57) ABSTRACT

The invention provides methods for producing live, attenuated viral vaccines, such as flavivirus vaccines.

This application claims priority from U.S. Provisional Patent Application Serial No. 60/348,565, filed Jan. 15, 2002.

### FIELD OF THE INVENTION

This invention relates to methods for producing viral vaccines.

### BACKGROUND OF THE INVENTION

Vaccines have been developed for the prevention of several significant human diseases of viral origin. Viral vaccines can generally be divided into the following three groups: (i) live, attenuated, (ii) inactivated, and (iii) subunit, based on the nature of the active agents of the vaccines. Each of these types of vaccines has its own distinct advantages and manners of production. Live, attenuated vaccines, for example, simulate natural infections and thus stimulate long-lasting antibody production, induce a good cell-mediated response, and induce resistance at the point of entry. These vaccines have generally been produced in primary cell lines, chick embryos, and diploid cell lines.

Inactivated or killed vaccines, in contrast, typically stimulate only a brief immune response, and thus require periodic boosting. In addition, local resistance at the point of entry, as well as cell-mediated immune responses, are poor with inactivated vaccines. An advantageous feature of these vaccines is that a robust method of producing them, employing heteroploid Vero cells, has been developed. An advantage of this production system is that Vero cells can be grown on beads, thus facilitating large-scale production. Additionally, in contrast to other cell types used in vaccine production, Vero cells do not produce interferon, which inhibits viral replication. Use of the Vero cell system, however, requires extensive purification, to eliminate from vaccine preparations potentially tumorigenic DNA derived from the heteroploid Vero cells. Inactivated vaccines can withstand such purification, because, in contrast to live vaccines, maintenance of infectivity is not required to achieve a sufficiently safe level of purity.

Flaviviruses are members of a family of small, enveloped positive-strand RNA viruses, some of the members of which pose current or potential threats to global public health. For example, Japanese encephalitis is a significant public health problem involving millions of at risk individuals in the Far East. Dengue virus, with an estimated annual incidence of 100 million cases of primary dengue fever and over 450,000 cases of dengue hemorrhagic fever worldwide, has emerged as the single most important arthropod-transmitted human disease.

Other flaviviruses continue to cause endemic diseases of variable nature and have the potential to emerge into new areas as a result of changes in climate, vector populations, and environmental disturbances caused by human activity. These flaviviruses include, for example, St. Louis encephalitis virus, which causes sporadic, but serious, acute disease in the midwest, southeast, and western United States; West Nile virus, which causes febrile illness, occasionally complicated by acute encephalitis, and is widely distributed throughout Africa, the Middle East, the former Soviet Union, and parts of Europe; Murray Valley encephalitis virus, which causes endemic nervous system disease in Australia; and Tick-borne encephalitis virus, which is distributed throughout the former Soviet Union and eastern Europe, where its Ixodes tick vector is prevalent and responsible for a serious form of encephalitis in those regions.

Hepatitis C virus (HCV) is another member of the flavivirus family, with a genome organization and a replication strategy that are similar, but not identical, to those of the flaviviruses mentioned above. HCV is transmitted mostly by parenteral exposure and congenital infection, is associated with chronic hepatitis that can progress to cirrhosis and hepatocellular carcinoma, and is a leading cause of liver disease requiring orthotopic transplantation in the United States.

Fully processed, mature virions of flaviviruses contain three structural proteins, envelope (E), capsid (C), and membrane (M), and seven non-structural proteins (NS1, NS2A, NS2B, NS3, NS4A, NS4B, and NS5). Immature flavivirions found in infected cells contain pre-membrane (prM) protein, which is the precursor to the M protein. The flavivirus proteins are produced by translation of a single, long open reading frame to generate a polyprotein, and a complex series of post-translational proteolytic cleavages of the polyprotein by a combination of host and viral proteases, to generate mature viral proteins (Amberg et al., J. Virol. 73:8083-8094, 1999; Rice, “Flaviviridae,” In Virology, Fields (ed.), Raven-Lippincott, New York, 1995, Volume I, p. 937). The virus structural proteins are arranged in the order C-prM-E. These proteins are present in the N-terminal region of the polyprotein, while the non-structural proteins (NS1, NS2A, NS2B, NS3, NS4A, NS4B, and NS5) are located in the C-terminal region of the polyprotein. The amino termini of prM, E, NS1, and NS4B are generated by host signalase cleavage within the lumen of the endoplasmic reticulum (ER), while most cleavages within the non-structural region are mediated by a viral protease complex known as NS2B-NS3 (Rice, supra). In addition, the NS2B-NS3 protease complex is responsible for mediating cleavages at the C terminus of both the C protein and the NS4A protein (Amberg et al., supra).

Because of the current and potential impacts of flaviviruses on global public health, the development of vaccines against flaviviruses has been a focus of significant research efforts. One approach to vaccinating against flavivirus infection has involved the construction of live, attenuated chimeric flaviviruses. In one example of such a chimera, the prM and E proteins of one flavivirus (e.g., yellow fever virus) are replaced by the prM and E proteins of another flavivirus (e.g., Japanese encephalitis, dengue, or West Nile virus), to which immunity is sought. Details of the construction and use of such chimeras in vaccination methods are provided, for example, in International applications PCT/US98/03894 and PCT/US00/32821, and in Chambers et al., J. Virol. 73:3095-3101, 1999.

### SUMMARY OF THE INVENTION

The invention provides methods of producing vaccines containing live, attenuated viruses (e.g., flaviviruses, such as a yellow fever virus, or chimeric viruses). These methods involve (i) introducing a nucleic acid molecule corresponding to the genome of the virus into heteroploid cells (e.g., Vero cells); (ii) treating virus harvested from the cells with a nuclease (e.g., Benzoase®); and (iii) formulating the nuclease-treated virus for administration as a vaccine. Further, the methods can, optionally, include the step of concentrating the virus after treatment with the nuclease.

Optionally, the virus produced using these methods is a chimeric flavivirus. Such a chimeric flavivirus may include, for example, (i) a yellow fever virus in which the nucleotide sequence encoding a prM-E protein is either deleted, truncated, or mutated so that functional yellow fever virus prM-E protein is not expressed, and (ii) integrated into the genome of the yellow fever virus, a nucleotide sequence encoding a prM-E protein of a second, different flavivirus, so that the prM-E protein of the second flavivirus is expressed. The second flavivirus may be, for example, Japanese Encephalitis virus, a Dengue virus (e.g., one of Dengue types 1, 2, 3, and 4), a Murray Valley Encephalitis virus, a St. Louis Encephalitis virus, a West Nile virus, a Tick-borne Encephalitis virus (e.g., Central European Encephalitis virus or Russian Spring-Summer Encephalitis virus), a Hepatitis C virus, a Kunjin virus, a Powassan virus, a Kyasanur Forest Disease virus, or an Omsk Hemorrhagic Fever virus.

In the chimeric viruses used in the invention, the nucleotide sequence encoding the prM-E protein of the second, different flavivirus preferably replaces the nucleotide sequence encoding the prM-E protein of the yellow fever virus. Also, the prM signal of the chimeric virus is, preferably, that of yellow fever virus.

The invention also includes vaccine compositions that are prepared using the methods described herein.

The invention provides several advantages. For example, as is noted above, heteroploid cells such as Vero cells enable the large-scale production of viral vaccines, as they can be grown on beads, and thus can be produced in large (e.g., 1,000-2,000 L) biofermentors. In addition, these cells do not produce interferon, which inhibits viral replication. Further, the number of purification steps in the methods of the invention are few, as compared to some other vaccine production methods, thus leading to decreased production costs.

Other features and advantages of the invention will be apparent from the following detailed description, the drawings, and the claims.

### BRIEF DESCRIPTION OF THE DRAWINGS

FIG. 1 is a flow chart diagram of a process for manufacturing a viral vaccine using the methods of the invention.

FIG. 2 is a flow chart diagram of a method for preparing RNA that can be used in the initial transfection of the methods of the invention.

FIG. 3. is a schematic representation of plasmids that can be used in the construction of a chimera that includes cloned yellow fever and JE (SA-14-14-2) virus sequences, as well as a method of using these plasmids to make such a chimera.

FIG. 4. is a flow chart diagram of a method for producing ChimeriVax™-JE Passage (1) and Passage (2).

FIG. 5 is a flow chart diagram of a method for producing a Master Seed (P3).

FIG. 6 is a flow chart diagram of a method for producing a Production Seed (P4).

FIG. 7 is a flow chart diagram of a method for producing a Final Bulk Vaccine (P5).

FIG. 8 is a flow chart diagram of a method of producing a Final Filled Vaccine (P5).

### DETAILED DESCRIPTION

The invention provides methods for producing vaccines that contain live, attenuated viruses. These methods can be used, for example, in the production of flavivirus vaccines (e.g., chimeric flavivirus vaccines). In general, the methods of the invention involve the introduction of a nucleic acid molecule (e.g., an RNA molecule) corresponding to the genome of a virus into heteroploid cells (e.g., Vero cells), harvesting virus from the medium in which the cells have been cultured, treating virus obtained in this manner with a nuclease (e.g., an endonuclease that degrades both DNA and RNA, such as Benzonase™; U.S. Pat. No. 5,173,418), concentrating the nuclease-treated virus (e.g., by use of ultrafiltration using a filter having a molecular weight cut-off of, e.g., 500 kDa), and formulating the concentrated virus for the purposes of vaccination. Details of the methods of the invention are provided below, after a description of viruses that can be the active ingredients of the vaccines made by these methods.

The methods of the invention can be used to produce vaccines containing any type of live, attenuated virus. Preferably, the virus is a live, attenuated flavivirus. For example, vaccine containing a live, attenuated yellow fever virus (e.g., YF17D) can be produced. Other examples of viruses that can be included in vaccines produced using the methods of the invention are listed below, in reference to chimeric viruses. In addition to being used as agents for inducing immunity against themselves, these viruses can also be used as vectors for carrying heterologous antigens, such as antigens from other pathogens. Vaccines containing chimeric flaviviruses, which are described further below, can also be produced using the methods of the invention.

Chimeric viruses that can be included in the vaccines that are made using the methods of the invention can consist of a flavivirus (i.e., a backbone flavivirus) in which a structural protein (or proteins) has been replaced with a corresponding structural protein (or proteins) of a second virus (i.e., a test or a predetermined virus, such as a flavivirus). For example, the chimeras can consist of a backbone flavivirus in which the prM and E proteins of the flavivirus have been replaced with the prM and E proteins of the second, test virus.

The chimeric viruses that are present in the vaccines produced using the methods of the invention can be made from any combination of viruses. Preferably, the virus against which immunity is sought is the source of the inserted structural protein(s). Examples of particular flaviviruses that can be used in the invention, as backbone or insert viruses, include mosquito-borne flaviviruses, such as Japanese encephalitis, Dengue (serotypes 1-4), Yellow fever, Murray Valley encephalitis, St. Louis encephalitis, West Nile, Kunjin, Rocio encephalitis, and Ilheus viruses; tick-borne flaviviruses, such as Central European encephalitis, Siberian encephalitis, Russian Spring-Summer encephalitis, Kyasanur Forest Disease, Omsk Hemorrhagic fever, Louping ill, Powassan, Negishi, Absettarov, Hansalova, Apoi, and Hypr viruses; as well as viruses from the Hepacivirus genus (e.g., Hepatitis C virus). Additional viruses that can be used as the source of inserted structural proteins include viruses from the Pestivirus genus (e.g., Bovine diarrhea virus), and other viruses, such as Lassa, Ebola, and Marburg viruses.

A specific example of a chimeric virus that can be included in the vaccines produced using the methods of the invention is the yellow fever human vaccine strain, YF17D, in which the prM and E proteins have been replaced with prM and E proteins of another flavivirus, such as Japanese encephalitis virus, West Nile virus, St. Louis encephalitis virus, Murray Valley encephalitis virus, a Dengue virus, or any other flavivirus, such as one of those listed above. For example, the following chimeric flaviviruses, which were deposited with the American Type Culture Collection (ATCC) in Manassas, Va., U.S.A. under the terms of the Budapest Treaty and granted a deposit date of Jan. 6, 1998, can be used in the invention: Chimeric Yellow Fever 17D/Dengue Type 2 Virus (YF/DEN-2; ATCC accession number ATCC VR-2593) and Chimeric Yellow Fever 17D/Japanese Encephalitis SA14-14-2 Virus (YF/JE A1.3; ATCC accession number ATCC VR-2594).

Details of making chimeric viruses that can be used in the invention are provided, for example, in International applications PCT/US98/03894 and PCT/US00/32821; and Chambers et al., J. Virol. 73:3095-3101, 1999, each of which is incorporated by reference herein in its entirety.

Details of a purification method of the invention are now provided, using a YF/JE chimera as an example. This method, as well as variants of this method, can be used to produce a vaccine containing any live, attenuated virus, such as the other chimeras described herein.

Overview of the Method

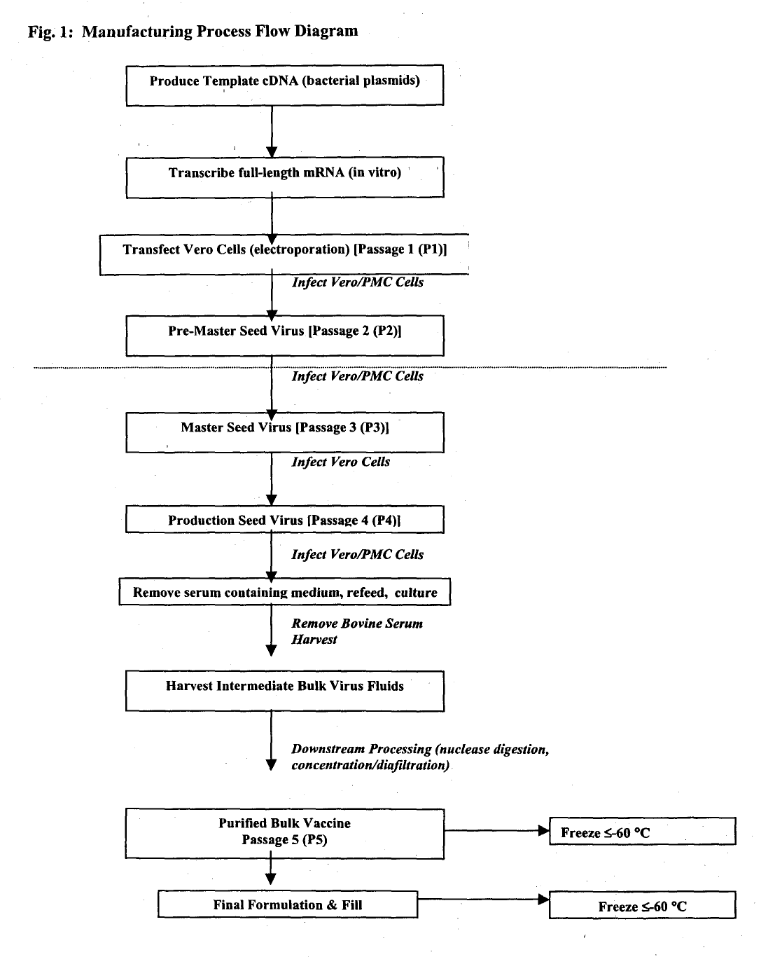

A simplified flow diagram of the manufacturing process is presented in FIG. 1, and is described in overview as follows.

A full-length cDNA template of yellow fever 17D in which the genes encoding the structural proteins (prM and E) YF 17D are replaced with the corresponding genes of an attenuated JE SA14-14-2 virus was used as a template for RNA synthesis. Full-length RNA was then synthesized using SP6 polymerase. Since flaviviruses have positive-sense RNA (i.e., they are infectious and serve as message for translation of all of the proteins required for replication), the chimeric RNA transcripts can be transfected into cells, which then produce infectious progeny virus.

The manufacturing process is initiated using RNA transcribed from full-length plasmid cDNA. The RNA solution (in SP6 polymerase reaction mixture (Epicentre Technologies; Madison, Wis.)) is used to transfect Vero/PMC cells by means of electroporation (cell passage 1). Progeny virus (passage 1) contained in cell culture medium is amplified by passage in Vero/PMC cells at a limiting dilution MOI (Multiplicity of Infection) of 4.965×10⁻⁷ (passage 2; Pre-Master Seed). Two further sequential passages (passage 3 and 4) are made at an MOI of 0.001 in cultures of Vero/PMC cells grown in Nunc cell factories (NCFs) in order to produce the Master and Production Virus Seeds, respectively. Fetal bovine serum (FBS) from a U.S. approved source, which is always heat-inactivated prior to use in any steps, is incorporated in the growth Minimum Essential Medium Earle's Salt (MEME) with L-glutamine, 1 mM sodium pyruvate, and 10% heat inactivated FBS. Master and Production Seeds are prepared from maintenance medium harvested 4 days post-infection with the virus and clarified by large pore (0.45 μm) filtration to remove cell debris. For storage of the Master and Production Seeds, the concentration of FBS is increased to 50% v/v, and the seeds are frozen at ≦−60° C.

A vaccine lot (passage 5) is produced under similar conditions of growth, except that the cell cultures used for propagating virus are rinsed prior to infection to remove fetal bovine serum, and maintenance medium without FBS or phenol red is added. The serum free medium containing virus is harvested on day 3 post-infection. The harvests are filtered at 0.22 μm to remove cell debris, and are then subjected to nuclease digestion and concentration/diafiltration to produce the Final Bulk. The Final Bulk is stabilized with 7.5% Lactose, NF, then it is sterile-filtered, normal Human Serum Albumin (HSA, USP) is added to a final concentration of 2.5%, and the Bulk is filled into the bulk containers, and frozen at ≦−60° C. Following determination of virus titer, the bulk is diluted with MEME with 7.5% lactose and 2.5% HSA, USP, without phenol red or L-glutamine. Then it is filled into final containers, labeled and stored frozen at ≦−60° C.

Vero/PMC cells were obtained from Aventis-Pasteur, formerly Pasteur-Mérieux-Connaught (PMC). The Vero/PMC continuous cell line was originally isolated and established by Yasumura et al. (Nihon Rinsho 21, 1201-1215, 1963). The original cells were taken from kidney cells of the African green monkey (Cercopithecus aethiops). This bank of cells is held by the American Type Culture Collection (Manassas, Va.) under reference ATCC-CCL81. The Mérieux Institute received one ampoule of these cells at the 124^{th} passage. The cells were amplified from the 124^{th} passage to the 129^{th} passage in Medium 199 (Earle) supplemented with FBS and thus formed the Primary Cell Bank. Cell material from the Primary Cell Bank underwent serial subculture and was then combined into a homogenous pool to constitute the Working Cell Bank.

Cell material from the Working Cell Bank underwent serial subculture (4 passages) to produce the cells that we used (OraVax Lot #R022399B) in the preparation of Pre-Master Master and Production Seeds. Vero cells used for production of Bulk vaccine were from OraVax Lot #C082599A. Vero/PMC cells from a bank prepared by PMC were used in the preparation of the passage 5 vaccine bulk. MEME supplemented with FBS and antibiotics was used in the passage of cells for the Working Cell Bank and the cells provided to us. The lot of Vero/PMC cells obtained was used in the manufacturing operations described in below with minimal further passaging with FBS, but without antibiotics. The lot was tested for identity by Isoenzyme Analysis, and was found to contain Cercopithecus monkey cells with no evidence of other cells. These cells were also found to be sterile and free of mycoplasma.

All animal and human derived proteins used in this process, including fetal bovine serum, trypsin, and human serum albumin, are from qualified U.S. sources. In addition, a Quality Control testing program is applied to the cells, seed viruses, and vaccine, which conforms to current recommendations for testing animal cell lines and products produced in them.

Details of YF/JE Chimera

We have developed a vaccine indicated for the prevention of Japanese encephalitis (JE) in persons residing in, or traveling to, areas of Asia and Australia endemic for this disease and for laboratory workers exposed to JE virus. The vaccine candidate ChimeriVax™-JE is a live, attenuated, genetically-engineered virus prepared by replacing the genes encoding two structural proteins (prM and E) of yellow fever 17D vaccine virus with the corresponding genes of an attenuated strain of JE virus (Chambers et al., 1998). The prM and E proteins of JE virus contain the critical antigens conferring protective humoral and cellular immunity, as shown by many previous studies with recombinant subunits, poxvirus vectors, and DNA vaccines (Konishi et al., Virology 185(1):401-410, 1991; Konishi et al., Virology 190(1):454-458, 1992; Konishi et al., Virology 188(2):714-720, 1992; Konishi et al., J. Virol. 72(6):4925-4930, 1998; Mason et al., Virology 180(1):294-305, 1991).

The JE prM and E genes in the chimeric vaccine candidate were derived from the JE SA14-14-2 strain (a live, attenuated vaccine strain licensed for use in China) because the biological properties and molecular characterization of this strain are well documented. The genetic rearrangement was accomplished by standard cloning techniques, employing two bacterial plasmids containing cDNA copies of the prM-E genes of JE SA14-14-2 virus and the remaining genes of yellow fever 17D. A full-length ‘chimeric’ yellow fever-JE infectious cDNA clone was generated.

To prepare the vaccine for clinical use, Vero/PMC cells are transfected by electroporation with RNA transcribed from the full-length cDNA clone. The progeny virus is amplified by one passage in Vero/PMC cells. Two further passages in Vero/PMC cells are made to produce the Master and Production Seeds, respectively. The Production Seed is used to produce vaccine lots, which are never more than five in vitro passages from the original RNA transcript. The Vero/PMC cell bank used for the production of Pre-Master, Master and Production Seeds is obtained directly from Aventis-Pasteur formally Pasteur-Merieux-Connaught. Cells are passaged in MEME supplemented with L-glutamine, 1 mM sodium pyruvate, and 10% heat inactivated FBS.

The vaccine lot is produced by us in Vero/PMC cells obtained from PMC. The vaccine lot is prepared from cell culture supematent fluid, which consists of MEME with 2 mM L-glutamine, without phenol red or FBS. The virus is treated with Benzonase® to remove host cell DNA and is partially purified by ultrafiltration. For initial clinical trials, the vaccine will be prepared as a partially purified, frozen formulation in MEME with 7.5% lactose, NF and 2.5% HSA, USP without phenol red or L-glutamine. The vaccine will be diluted immediately before use with MEME containing 7.5% Lactose, NF and 2.5% HSA, USP to a final concentration of either 1×10⁵ pfu/mL (high dose) or 1×10³ pfu/mL (low dose). The vaccine will be administered by subcutaneous injection.

Materials Used in Manufacture

Table 1 lists the raw materials used in the production of drug substance. All media, buffers and reagents, which are exposed to the product, are tested for the absence of microbial contamination.

TABLE 1
Drug Substance Raw Materials

| Raw MateriaI | Grade | Supplier |
|---|---|---|
| Defined Fetal Bovine Serum, | US Sourced | Hyclone |
| 40 nm filtered | | Logan, UT |
| Minimal Essential Medium | ACS | Life Technologies |
| (1X), liquid with Earle's Salt | | Rockville, MD |
| with L- | | |
| glutamine | | |
| MEM Sodium Pyruvate | ACS | Life Technologies |
| Solution, 100 mM (100X), | | |
| liquid 11.004 mg/L | | |
| Lactose, Monohydrate, Powder, | N. F. | J.T. Baker |
| | | Phillipsburg, NJ |
| Minimum Essential Medium | ACS | Life Technologies |
| (1X), liquid with Earle's salts | | Rockville, MD |
| w/o L-glutamine w/o phenol red | | |
| Benzonase ® purity | ACS | American Interna- |
| grade I 500,000 U/vial for | | tional Chemical |
| biotechnology | | Natick, MA |
| Trypsin-Versene Mixture | Cell Culture Grade | BioWhittaker |
| | | Walkersville, MD |
| Dulbecco's Phosphate Buffered | ACS | Life Technologies |
| Saline (1X), liquid without | | Rockville, MD |
| calcium chloride without | | |
| magnesium chloride | | |
| PBS w/o Calcium or Magnesium | Cell Culture Grade | BioWhittaker |
| | | Walkersville, MD |
| L-glutamine 200 mM in 0.85% | Cell Culture Grade | BioWhittaker |
| NaCl Solution | | Walkersville, MD |
| Human Serum Albumin, 20% 50 | USP | Instituto Grifols |
| mL 10.0 g | | Barcelona, Spain |
| Water for Injection | USP | Baxter Healthcare |
| | | Corporation |

Production of Template DNA for in vitro Transcription of ChimeriVax™-JE

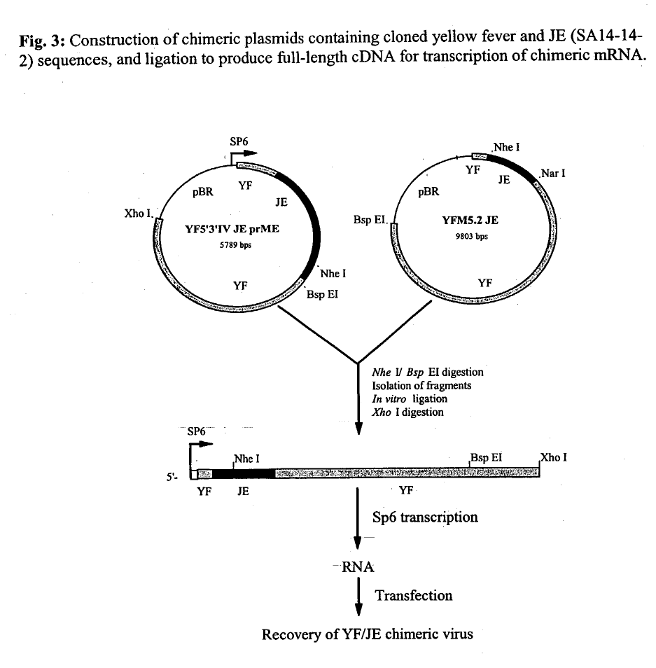

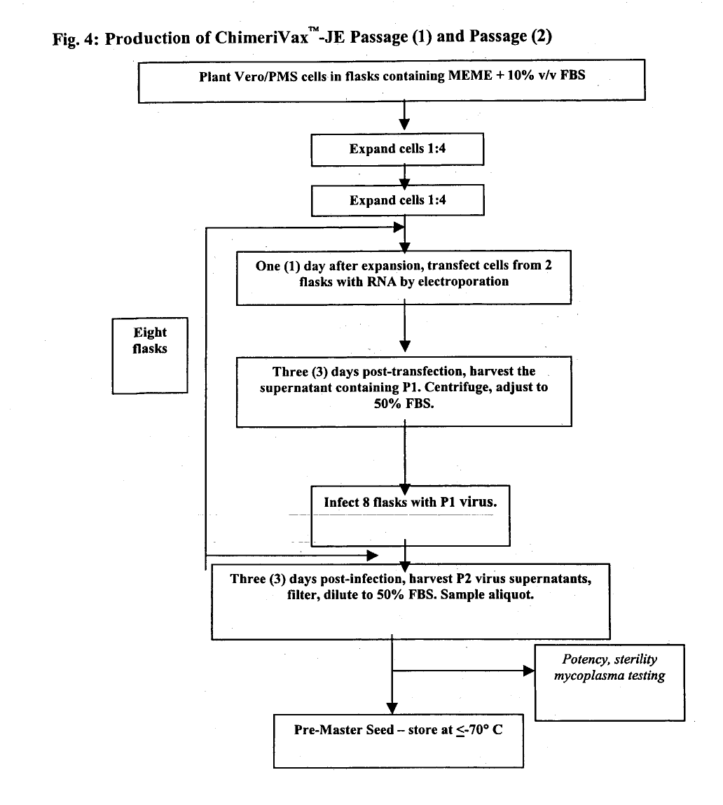

A flow chart detailing the preparation of the RNA used in the initial transfection is given in FIG. 3.

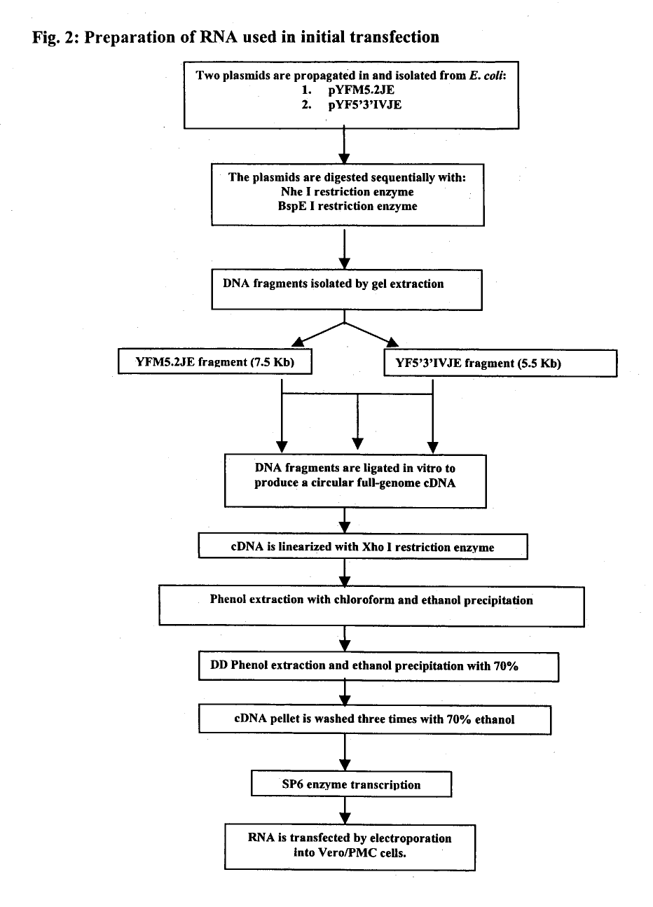

YF 17D genomic sequences are propagated in two plasmids (YF5′3′IV JE PrME and YFM5.2 JE), which encode the YF sequences from nucleotide (nt) 1-2276 and nt 8279-10,861 (YF5′3′IV JE PrME) and from nt 1373-8704 (YFM5.2), respectively (FIG. 2). Full-length cDNA templates are generated by ligation of appropriate restriction fragments derived from these plasmids. Construction of chimeric virus involves replacement of YF sequences within the YF5′3′IV JE PrME and YFM5.2 JE plasmids by the corresponding JE sequences from the start of the prM protein (nt 478, amino acid (aa) 128) through the E/NS1 cleavage site (nt 2452, aa 817) (Chambers et al., J. Virol. 73(4):3095-3101, 1999; Rice et al., New Biol. 1:285-296, 1989). Appropriate restriction sites were engineered in both the YF and JE sequences to permit in vitro ligation. The structure of the template for regenerating chimeric YF/JE virus is shown in FIG. 2.

The structural regions of JE SA14-14-2 virus are cloned in two pieces that overlap at an NheI site (JE nt 1125), which can then be used for in vitro ligation. Methods for the generation of full-length cDNA templates, RNA transfection and recovery of infectious virus are essentially as described previously (Chambers et al., J. Virol. 73(4):3095-3101, 1999). Prior to in vitro ligation to obtain full-length cDNA, plasmids YFM5.2 and YF5′3′IV are double digested by restriction endonucleases NheI and BspEI. Next, ChimeriVax™-JE fragments are gel purified and ligated with T4 DNA ligase in a 2:1 ratio of insert:vector (900 ng total DNA per reaction). The ligation product is linearized with XhoI restriction endonuclease. Full length DNA template is then chloroform extracted to remove phenol traces, ethanol precipitated and desalted with 70% ethanol. The resulting pellet is resuspended in RNAse free water.

Transcription of Full-Length Messenger RNA for GMP Production

Transcription is performed with 225 ng of XhoI-linear cDNA template (see FIG. 2) and using the protocol for capped RNA as recommended for the AmpliScribe™ SP6 Transcription Kit (Epicentre Technologies, Madison, Wis.). Final reaction volume is 20 μl and the reaction mixture includes: GTP (1.5 mM), ATP, CTP, UTP (7.5 mM each), 10× Kit Reaction Buffer (1×), 20 mM methylated CAP Analog (3 mM), 100 mM DTT (10 mM), 0.5 μl of recombinant Rnasin® (Promega, Madison, Wis.), and 2.0 μl of AmpliScribe Enzyme Solution. The reaction mixture is incubated at 40° C. for 2 hours and then stored at minus 70° C. RNA yields are estimated by gel analysis of a reaction aliquot.

Virus Seed Production

Expansion of Vero/PMC Cells for Production of Passage 1 and Passage 2 Virus

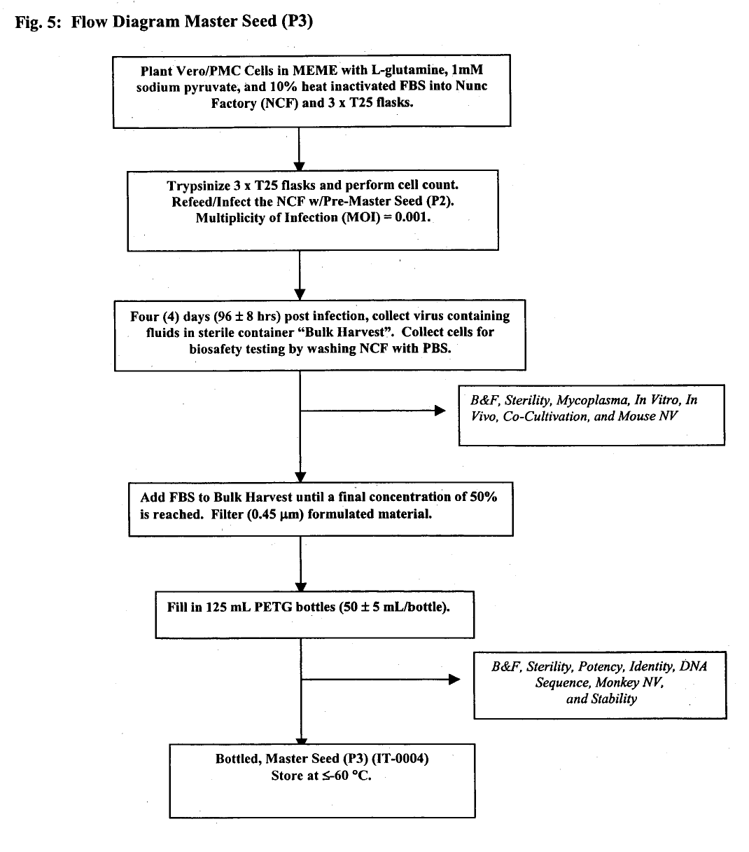

A flow diagram depicting the process used in the preparation of passage 1 (P1) and passage 2 (P2) virus is shown in FIG. 4. The process involves a biomass expansion phase in which the numbers of Vero/PMC cells are expanded using stationary culture flasks prior to transfection with RNA or infection with virus.

The biomass expansion is carried out in stationary culture using 150 cm² (T150) cell culture flasks. Cell growth medium consists of MEME supplemented with fetal bovine serum at a final concentration of 10% v/v and 1.0 mM sodium pyruvate. A vial of Vero/PMC cells is removed from liquid nitrogen vapor storage and thawed rapidly at 37° C. The vial is transferred to a Class II Laminar Flow biological safety cabinet (BSC) and the exterior surface disinfected with filtered 70% ethanol. The contents of the vial are carefully removed and warm cell growth medium is slowly added to the cells. The volume of the cell suspension is adjusted to 200 mL with additional cell growth medium and 100 mL of the cell suspension is added to each of two T150 flasks. The flasks are capped and placed in a 37° C., 5% CO₂ incubator.

Three days after the initial plant, the flasks containing the cells are removed from the incubator and examined microscopically. The monolayers are typically >60% confluent at this stage of the growth phase. The flasks are then taken to a BSC and the spent culture medium is removed. Twenty-five mL of Dulbecco's Phosphate Buffered Saline (PBS) are added to each flask and the cells are rinsed to remove excess serum. Ten mL of warm trypsin (0.25% of a 1:250 preparation) are added to each flask and the flasks are rocked to ensure that the entire surface is exposed to the trypsin. The flasks are then incubated at 37° C. for 10-15 minutes to dislodge the cells from the growth surface.

Fifteen mL of cell growth medium are added to each flask to neutralize the trypsin and the cell suspensions from each flask are pooled. A sample of the cell suspension is removed for a cell count. The volume of the cell suspension is adjusted to 800 mL with cell growth medium, and 100 mL of the cell suspension is added to each of eight T150 flasks. The flasks are placed in a 37° C., 5% CO₂ incubator.

Two days after the first passage, the cells are expanded 1:4 again (see description above). Because only a portion of the cells will be required for the infection with P1 virus, only four flasks are selected for the 1:4 expansion. The procedures used for this cell passage are the same as those described above.

Transfection of Vero/PMC Cells

Confluent cell monolayers are trypsinized, washed with RNAse-free PBS and resuspended in PBS at a density range of 6×10⁶ to 1.2×10⁷ cells/mL. A volume containing 250 ng of RNA transcript from the SP6 polymerase reaction (FIG. 3) is inoculated into a 0.4 cm gap electroporation cuvette followed by 500 μl of the Vero/PMC cell suspension. Cells are electroporated at 1200 V, 25 μF with the Gene Pulser II system (Bio-Rad Laboratories, Hercules, Calif.). After RNA electro-transfection, cells are transferred to a 25 cm² flask and allowed to recover in growth medium. After 24 hours, cells have restored to a monolayer of maximum confluency and growth medium is changed to reduce the FBS concentration to 3%. Virus is harvested on day 3 post-transfection by supernatant aspiration, and then are briefly centrifuged to remove cell debris. FBS to a 50% v/v final concentration is added as a stabilizer.

Pre-Master Seed (P2)

Four days after the second passage, the cells from eight of the flasks are infected with P1 virus, which has been harvested that same day. Virus-containing cell culture supernatants are removed from the flasks of cells that had been electroporated with ChimeriVax™-JE RNA three days previously. The supernatants are centrifuged briefly to remove cell debris and then diluted with an equal volume of heat-inactivated FBS. The eight flasks containing cells are removed from the incubator and the spent culture medium is removed. Diluted P1 virus is added to each flask in a small volume, the flasks recapped and the virus adsorbed to the cells for 1 hour at 37° C. After 1 hour, 100 mL of cell growth medium is added to each flask, the flasks are recapped and placed in a 37° C., 5% CO₂ incubator.

Three days after infection with P1 virus, the flasks are removed from the incubator and the culture fluids containing the P2 virus are harvested. The culture fluids are removed from the cultures, filtered to remove cell debris and diluted with an equal volume of heat-inactivated FBS. The diluted P2 virus is divided into smaller aliquots and the aliquots frozen at <−70° C.

Master Seed (P3)

A flow diagram depicting the process used in the preparation of the Master Seed (P3) Viruses is shown in FIG. 5. Vero/PMC cells are allowed to grow to near confluence in 10-layer NCFs, after which they are infected with virus. Virus-containing cell culture supernatant fluid is harvested and processed by filtration to remove cell debris.

The biomass expansion is carried out in a 10-tray Nunc Cell Factory (NCF). Growth medium consists of MEME with L-glutamine, 1 mM sodium pyruvate, and 10% heat inactivated FBS at a final concentration of 10% v/v and 1.0 mM sodium pyruvate. Approximately six days prior to the virus infection step, Vero/PMC cells from one vial are planted directly into a NCF. Prior to seeding, the NCF were conditioned for approximately 15 minutes with sterile filtered 5% CO₂. A cell suspension is made in approximately 3100 mL of MEME with L-glutamine, 1 mM sodium pyruvate, and 10% heat inactivated FBS. Approximately 3000 mL of the cell suspension is used to seed the NCF and three T25 flasks are seeded with 12.5 mL each. The seeding density is approximately 1.5×10⁴ cells/cm² for both flasks and NCFs. The NCF and three flasks are incubated at 36±2° C., 5±2% CO₂, and 80±5% relative humidity.

Six days after the initial plant, the three flasks are inspected by microscope for confluency. If the flasks are at least 50% confluent, then the NCF is infected with the virus. If the flasks are less than 50% confluent, then the NCF is infected the following day. Prior to infection, the three flasks are trypsinized and the cells counted. The number of cells in the NCF is calculated using the density determined from the flasks. The amount of Virus Seed (P2) needed to infect the cells at a multiplicity of infection (MOI) of approximately 0.001 is determined by calculation. The Virus Seed (P2) is thawed at 36° C. and diluted in approximately 250 mL of MEME with L-glutamine, 1 mM sodium pyruvate, and 10% heat inactivated FBS. The spent medium is removed from the NCF and approximately 250 mL of the diluted virus is transferred into the NCF and allowed to equilibrate. The NCF is returned to the incubator and is gently rocked every 15 minutes for approximately one hour to facilitate virus absorption onto the cells.

Following the one hour absorption period, the NCF is removed from the incubator and approximately 3000 mL of MEME with L-glutamine, 1 mM sodium pyruvate, and 10% heat inactivated FBS is aseptically added. The infected NCF is incubated at 36±2° C., 5±2% CO₂, and 80±5% relative humidity for four days (96±8 hours).

Four days post-infection, the NCF is removed from the incubator and the infected culture medium containing the Master seed virus (passage 3) is aseptically harvested into a sterile container. After draining the culture medium containing Master seed virus (P3) from the NCF, approximately 500 mL of Dulbecco's PBS is added to the NCF to lift cells from the surface. The NCF is then drained, the cells concentrated, and diluted with culture medium as necessary to achieve the desired cell concentration for Quality Control testing. After sampling, the remaining bulk Master Seed harvest material is formulated with FBS to a final concentration of 50%. The formulated bulk Master Seed is clarified using a sterile Millipak 200, 0.45 μm cartridge filter. The filtered material is pooled into a sterile container and sampled prior to filling.

The Master Seed (P3) is filled into sterile bottles and is stored at ≦−60° C.

Production Seed (P4)

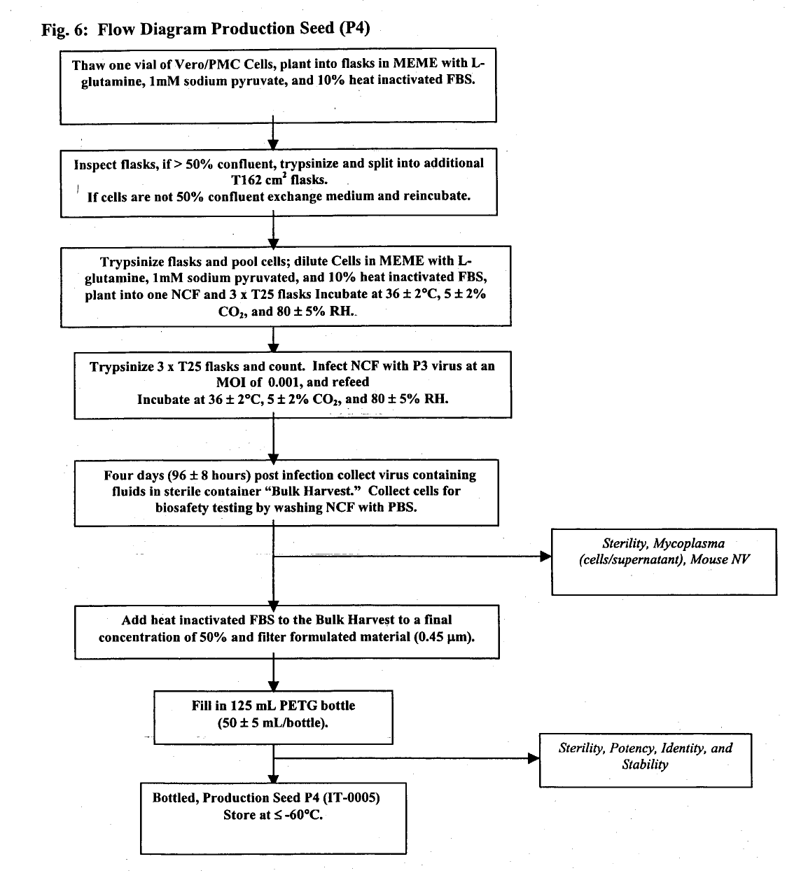

A flow diagram depicting the process used in the preparation of the Production Seed (P4) Virus is shown in FIG. 6.

One vial of Vero/PMC cells is thawed and cells are planted into six 162 cm² flasks (T162) at a seeding density of approximately 3-5×10⁴ cells/cm². Cells are incubated in MEME with L-glutamine, 1 mM sodium pyruvate, and 10% heat inactivated FBS for three days. On Day 3, the flasks are inspected. If the cells are greater than 50% confluent, then they are split into additional flasks at a seeding density of 1-5×10⁴ cells/cm². If the flasks are less than 50% confluent, then the medium is exchanged and the flasks are returned to the incubator. On Day 6, the flasks are trypsinized and the cells pooled. A cell count is performed. Cells are diluted in approximately 3100 mL of cell growth medium at a concentration of 3-10×10⁴ cells/mL. Approximately 3000 mL of the cell suspension are used to seed the NCF and three T25 flasks are seeded with 12.5 mL each. Prior to seeding, the NCF is conditioned for approximately 15 minutes with sterile filtered 5% CO₂. The seeding density in the NCF and in the flasks will be approximately 3-5×10⁴ cells/cm². The NCF and flasks are incubated at 36±2° C., 5±2% CO₂, and 80±5% relative humidity for four days (96±8 hours).

Passage 4 Production Seed (P4) is infected, harvested, formulated, and filled in the same manner as the Master Seed (P3).

Drug Substance Production

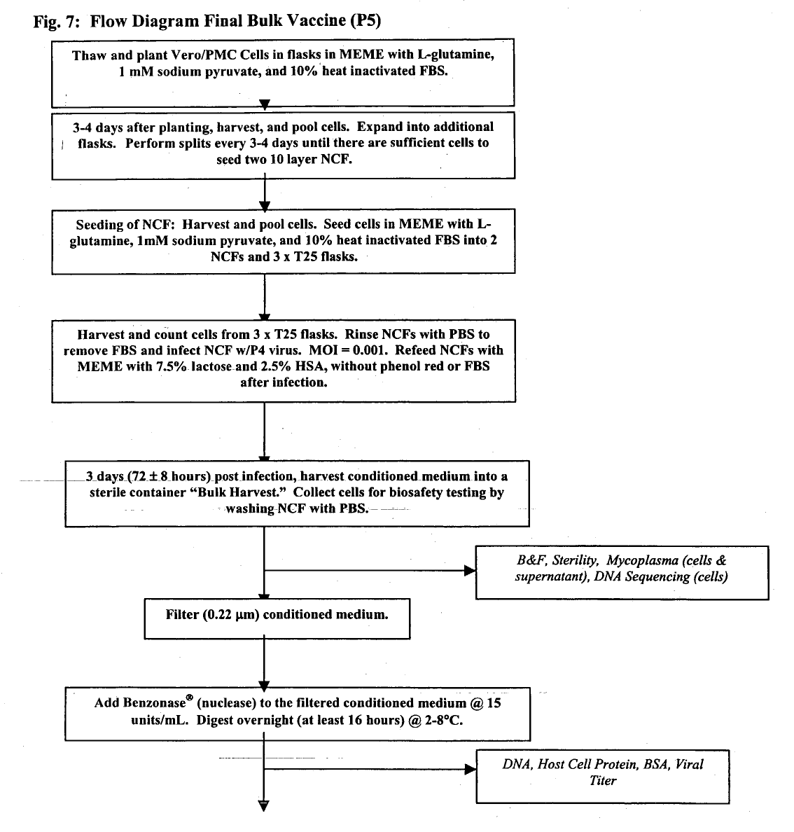

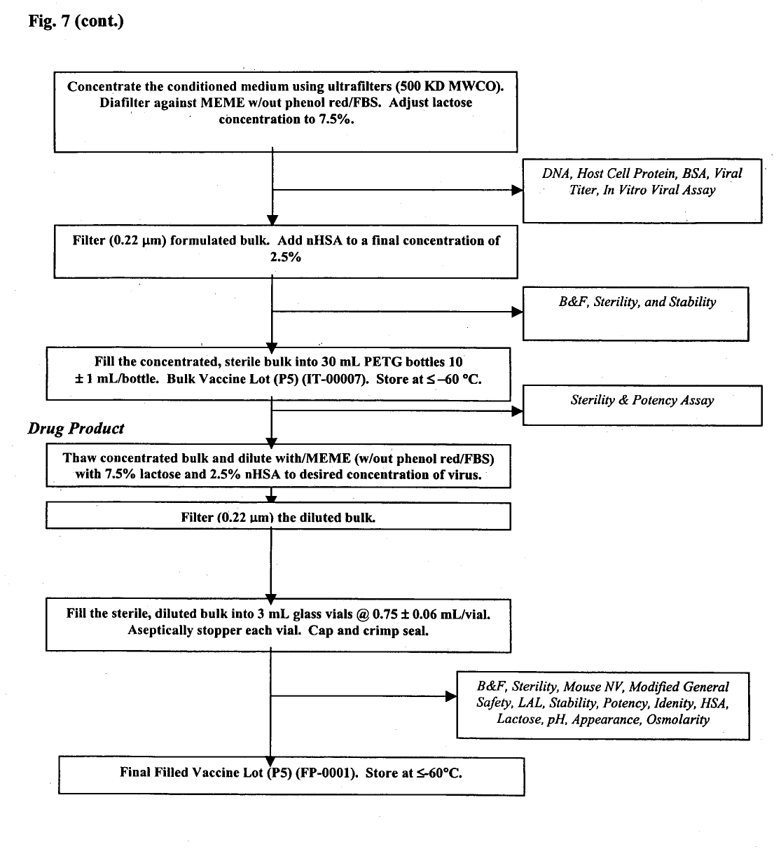

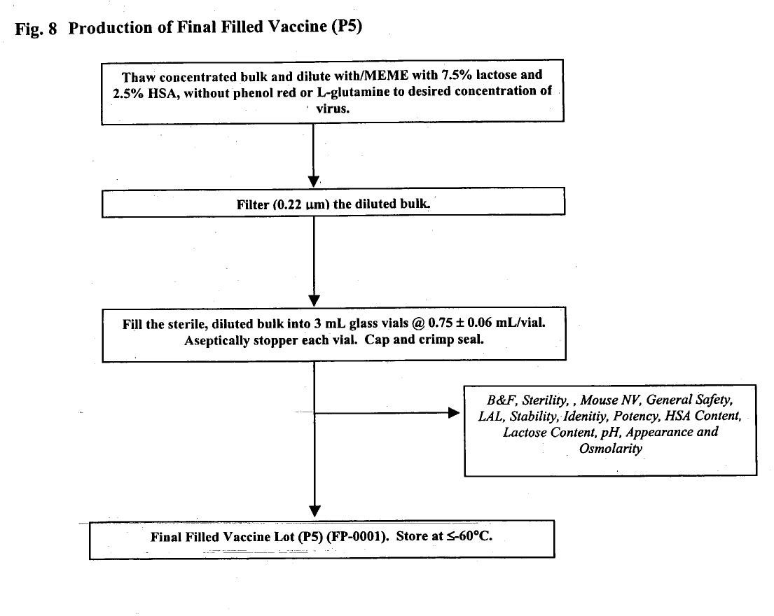

A flow diagram depicting the process used in the preparation of passage 5 (P5) Bulk vaccine virus using P4 virus is shown in FIG. 7, and the process used in the production of the Final Filled Vaccine is schematically illustrated in FIG. 8.

As with the processes used to prepare the P3 and P4 viruses, the process involves a biomass expansion phase in which the numbers of Vero/PMC cells are expanded using stationary culture flasks and NCFs. At the virus infection phase there is a wash step prior to virus infection and a refeed with serum-free medium, both of which are designed to reduce the levels of bovine serum albumin (BSA) in the final bulk. Downstream processing consists of filtration to remove cell debris followed by digestion of nucleic acids by Benzonase®, then the concentration of the virus and a final filtration.

The biomass expansion is carried out as described above. Vero/PMC cells grown in NCFs are infected with Production Seed (P4) virus at a multiplicity of infection (MOI) of approximately 0.001. The spent cell culture medium is removed from the NCFs and each NCF is rinsed twice with at least 250 mL of MEME with 2 mM L-glutamine, without phenol red or FBS. Production Seed virus (P4) diluted in a small volume of MEME with 2 mM L-glutamine, without phenol red or FBS is then pumped into the NCFs and allowed to adsorb for 1 hour. After the 1 hour adsorption, approximately 3000 mL of MEME with 2 mM L-glutamine without phenol red or FBS is aseptically added and the infected NCFs are incubated at 36±2° C., 5±2% CO₂, and 80±5% relative humidity for three days (72±8 hours).

Three days post-infection, the NCFs are removed from the incubator and the culture fluids containing the P5 virus are aseptically harvested into a sterile container. Approximately 250 mL of Dulbeccos PBS are added to each NCF to lift the cells from the surface. The NCFs are drained and the cells are pooled, concentrated, and diluted with culture medium. Representative samples are taken from the sterile container containing the Bulk Harvest for QC testing. The remaining harvest medium is clarified using a sterile Millipak 200, 0.22 μm cartridge filter.

Following the filtration step, Benzonase® (nuclease) is added to the unpurified conditioned medium at a concentration of 15 units/mL. The conditioned medium is refrigerated at 2-8° C. for at least 16 hours to allow for the digestion of nucleic acids.

Following Benzonase® overnight digestion, the unpurified conditioned medium is concentrated at room temperature to approximately 600 mL using Pellicon-2 Mini ultrafilter cassettes (500 kDa MWCO). The concentrate is then diafiltered against at least 3000 mL of MEME without phenol red or FBS. The concentrate is collected and the ultrafilters are rinsed with diafiltration medium into the concentrate in order to bring the final concentrate volume to 750 mL. The concentrate is then formulated by adding 67.5 grams of lactose. The volume is adjusted to approximately 800 mL with MEME without phenol red or FBS and is then filtered through a Millipak 40 filter cartridge (0.22 μm) into a sterile container. Following filtration, 110-115 mL of Human Serum Albumin (nHSA, USP 20%) is added, increasing the volume to approximately 900 mL.

The purified bulk vaccine lot (P5) is filled and stored at ≦−60° C.

Formulation and Use

Live, attenuated viruses that are produced using the methods of the invention can be formulated for use as vaccines using methods that are standard in the art. Numerous pharmaceutically acceptable solutions for use in vaccine preparation are well known in the art, and can readily be adapted for use in the present invention by those of skill in this art. (See, e.g., Remington's Pharmaceutical Sciences (18^{th} edition), ed. A. Gennaro, 1990, Mack Publishing Co., Easton, Pa.) However, the viruses can simply be diluted in a physiologically acceptable solution, such as sterile saline or sterile buffered saline.

Optionally, the vaccines can include an adjuvant or carrier, in addition to the live, attenuated virus. Adjuvants that can be used to enhance the immunogenicity of the chimeric vaccines include, for example, liposomal formulations, synthetic adjuvants, such as saponins (e.g., QS21), muramyl dipeptide, monophosphoryl lipid A, or polyphosphazine. Although these adjuvants are typically used to enhance immune responses to inactivated vaccines, they can also be used with live vaccines. In the case of a chimeric vaccine delivered via a mucosal route, for example, orally, mucosal adjuvants such as the heat-labile toxin of Escherichia coli (LT) or mutant derivations of LT are useful adjuvants. In addition, genes encoding cytokines that have adjuvant activities can be inserted into the viruses. Thus, genes encoding cytokines, such as GM-CSF, IL-2, IL-12, IL-13, or IL-5, can be inserted together with heterologous flavivirus genes, to produce a vaccine that results in enhanced immune responses, or to modulate immunity directed more specifically towards cellular, humoral, or mucosal responses.

The vaccines produced using the methods of the invention are administered in amounts, and by using methods, which can readily be determined by those of ordinary skill in this art. For example, the live, attenuated virus can be formulated as sterile aqueous solutions containing between 100 and 1,000,000 infectious units (e.g., plaque-forming units or tissue culture infectious doses) in a dose volume of 0.1 to 1.0 ml, to be administered by, for example, intramuscular, subcutaneous, or intradermal routes. In addition, because flaviviruses may be capable of infecting the human host via the mucosal routes, such as the oral route (Gresikova et al., “Tick-borne Encephalitis,” In The Arboviruses, Ecology and Epidemiology, Monath (ed.), CRC Press, Boca Raton, Fla., 1988, Volume IV, 177-203), the vaccine virus can be administered by a mucosal route to achieve a protective immune response. The vaccine can be administered as a primary prophylactic agent in adults or children at risk of flavivirus infection. The vaccines can also be used as secondary agents for treating flavivirus-infected patients by stimulating an immune response against the flavivirus.

What is claimed is:

1. A method of producing a vaccine comprising a live, attenuated virus, said method comprising the steps of:
introducing a nucleic acid molecule corresponding to the genome of said virus into heteroploid cells;
treating virus harvested from said cells with a nuclease; and
formulating the nuclease-treated virus for administration as a vaccine.

2. The method of claim 1, wherein said virus is a flavivirus.

3. The method of claim 2, wherein said flavivirus is a yellow fever virus.

4. The method of claim 1, wherein said virus is a chimeric virus.

5. The method of claim 4, wherein said chimeric flavivirus comprises a yellow fever virus in which the nucleotide sequence encoding a prM-E protein is either deleted, truncated, or mutated so that functional yellow fever virus prM-E protein is not expressed, and
integrated into the genome of said yellow fever virus, a nucleotide sequence encoding a prM-E protein of a second, different flavivirus, so that said prM-E protein of said second flavivirus is expressed.

6. The method of claim 5, wherein said second flavivirus is a Japanese Encephalitis virus.

7. The method of claim 5, wherein said second flavivirus is a Dengue virus selected from the group consisting of Dengue types 1, 2, 3, and 4.

8. The method of claim 5, wherein said second flavivirus is selected from the group consisting of a Murray Valley Encephalitis virus, a St. Louis Encephalitis virus, a West Nile virus, a Tick-borne Encephalitis virus, a Hepatitis C virus, a Kunjin virus, a Powassan virus, a Kyasanur Forest Disease virus, and an Omsk Hemorrhagic Fever virus.

9. The method of claim 8, wherein said Tick-borne Encephalitis virus is a Central European Encephalitis virus or a Russian Spring-Summer Encephalitis virus.

10. The method of claim 5, wherein the nucleotide sequence encoding the prM-E protein of said second, different flavivirus replaces the nucleotide sequence encoding the prM-E protein of said yellow fever virus.

11. The method of claim 5, wherein the prM signal of said chimeric virus is that of yellow fever virus.

12. The method of claim 1, wherein said heteroploid cells are Vero cells.

13. The method of claim 1, wherein said nuclease Benzoase®.

14. The method of claim 1, further comprising concentrating said virus after treatment with said nuclease.

15. A vaccine composition prepared using the method of claim 1.
